(19) United States
(12) Patent Application Publication
(10) Pub. No.: US 2014/0007956 A1
(43) Pub. Date: Jan. 9, 2014

(54) CATHETER AND METHOD

(71) Applicants: Guy Rutty, Leicester (GB); Sarah Saunders, Leicester (GB); Bruno Morgan, Leicester (GB); Vimal Raj, Leicester (GB)

(72) Inventors: Guy Rutty, Leicester (GB); Sarah Saunders, Leicester (GB); Bruno Morgan, Leicester (GB); Vimal Raj, Leicester (GB)

(73) Assignee: University of Leicester, Leicester (DE)

(21) Appl. No.: 13/985,663

(22) PCT Filed: Feb. 17, 2012
(86) PCT No.: PCT/GB12/50359

§ 371 Date: Sep. 11, 2013

(30) Foreign Application Priority Data
Feb. 18, 2011 (GB) 1102867.7

(51) Int. Cl. A61B 19/00

(52) U.S. Cl. CPC A61B 19/46
USPC 137/551

(57) ABSTRACT

This invention relates to a catheter for use in post-mortem angiography and a method for post-mortem in-situ cardiac angiography of a human or animal body. The catheter comprises (a) a proximal end for insertion into a human or animal body, (b) a distal end which remains outside the body during normal use, (c) a tube for delivering an imaging agent to the body, and (d) a marker for insertion into the body, the marker being positioned at a predetermined distance from the proximal end and comprising a substance such that its position once inserted into the body is detectable from outside the body.

This invention relates to a catheter for use in post-mortem angiography and a method for carrying out post-mortem angiography. The catheter and method are suitable for use on humans and or animals, of any age.

With the increasing use and availability of multi-detector computed tomography (MDCT) and magnetic resonance imaging (MRI) in autopsy practice, there has been an international push towards the development of the so called ‘near virtual autopsy’. However, currently a significant obstacle to the consideration as to whether or not near virtual autopsies could one day replace the conventional invasive autopsy is the failure of post-mortem imaging to yield detailed information concerning the coronary arteries. To date a cost effective, practical solution to allow high throughput imaging has not been presented within the forensic literature. This invention seeks to provide a simple, quick, cost effective, manual, targeted in-situ post-mortem cardiac angiography catheter and method using a minimally invasive approach, to be used with multi-detector computed tomography for high throughput cadaveric imaging which can be used in permanent or temporary mortuaries.

The use of computed tomography (CT) was first reported in association with post-mortem practice in 1983. Since then the use of multi-detector computed tomography (MDCT) and magnetic resonance imaging (MRI) has been widely reported within the forensic and radiological literature. Despite the advances in this field, a significant obstacle to the acceptance of so-called ‘near virtual autopsies’ relates to the diagnosis of cardiac death and the failure of standard post-mortem imaging to yield detailed information concerning the coronary arteries. This is often put forward as the principle reason why a ‘near virtual autopsy’ currently does not provide a realistic alternative to the invasive autopsy.

In clinical practice, cardiac MDCT including contrast enhanced CT coronary angiography is fast emerging as a powerful diagnostic tool for the assessment of coronary disease in both acute and chronic cases. Cardiac MDCT has been demonstrated to be excellent at identifying the course of anomalous coronary arteries, congenital heart disease, pericardial disease, cardiac masses, thrombi and aortic disease. The benefit of MDCT, compared to conventional angiography is that the artery wall can be visualised in addition to the lumen. The degree of wall calcification can be quantified (so-called ‘calcium scoring’) and used to stratify risk of future cardiac events. The implementation of cardiac MDCT coronary angiography in the post-mortem setting is impaired by the lack of an active circulation to deliver suitable contrast agents via an intravenous route. Thus vessel lumen and wall pathology, as well as course, are currently difficult to determine. To overcome this, a method of post-mortem MDCT coronary angiography must be used.

A number of studies have demonstrated the feasibility of post-mortem MDCT angiography in animals and humans. The main body of work has been performed on single organ systems, with imaging of the organ either by injection in situ and then removal for imaging, or injection of the organ after removal (i.e. invasive techniques). A handful of studies have looked at minimally invasive whole-body angiography. Whole body MDCT angiographic imaging was first published by the Virtopsy® group who, along with a number of other groups, have shown that post-mortem MDCT angiography is feasible. These methods are based on a complex system of whole body angiography using a modified heart and lung bypass machine. This method allows detailed assessment of the entire circulatory system which will have an important role in specific cases. However, if minimally invasive autopsy with angiography is to be implemented for routine coronial autopsies, the numbers of cadavers to be examined at centres will run into thousands, and such complex approaches under these circumstances may be impractical.

To date a cost effective method allowing high throughput cardiac CT imaging has not been presented within the forensic literature. This invention seeks to provide a simple, quick, cost effective, targeted in situ post-mortem cardiac angiography catheter and method using a minimally invasive approach, to be used with MDCT for high throughput cadaveric imaging.

According to one aspect of the invention there is provided a catheter for use in post-mortem angiography, the catheter comprising:
(a) a proximal end for insertion into a human or animal body,
(b) a distal end which remains outside the body during normal use,
(c) a tube for delivering an imaging agent to the body, and
(d) a marker for insertion into the body, the marker being positioned at a predetermined distance from the proximal end and comprising a substance such that its position once inserted into the body is detectable from outside the body.

The word “proximal” is used to refer to parts of the catheter that are closer to the end of the catheter that is to be inserted into the body. The word “distal” is used to refer to parts of the catheter that are closer to the end of the catheter that remains outside the body. The term “imaging agent” (or contrast agent) is used to refer to a substance (solid, liquid or gas) used to enhance the imaging (or contrast) of structures or fluids within a body. The term “angiography” is used to refer to the imaging of any part of the body, for example any organ or body cavity.

In some embodiments, the angiography is cardiac angiography.

In some embodiments, the tube has a diameter greater than or equal to 3 mm. In some embodiments the tube has a diameter greater than or equal to 4 mm. In some embodiments, the tube has a diameter of about 4.7 mm or about 14 Fr. In some embodiments, the tube is a 14Fr Foley catheter tube. The tube should have sufficient diameter to deliver the required amount of an imaging agent to the heart of the human or animal body on which the post-mortem angiography is carried out. Since the catheter is for use post-mortem, there is no blood circulation to assist in transporting the imaging agent (also known called contrast agent) around the body. This means that thinner catheter tubes such as those used to deliver imaging agent in cardiac catheterisation in live patients are not usable in post-mortem angiography.

In some embodiments, the tube comprises a flexible material, preferably a polymer. In some embodiments, the tube comprises a silicone polymer and/or latex. For example, tube may comprise latex coated with a silicone polymer. In some embodiments, the tube comprises a more rigid material such as a silicone polymer. In some embodiments, the tube comprises a polyolefin coating. Such a coating may be applied to the tube using a heat-shrink process. These coatings can be used to stiffen the tube. In some embodiments, medical grade polyolefin is used.

In some embodiments, the catheter is shaped to accept a guide wire which can extend along at least part of the length of the tube. In some embodiments, the catheter comprises a guide wire extending along at least part of the length of the tube. In some embodiments, the guide wire comprises a substance such that its position once inserted into the body is detectable from outside the body. In some embodiments, the guide wire comprises titanium. In addition to helping the user detect the position of the catheter, the guide wire can have the effect of enhancing the rigidity of the catheter. In some embodiments, if the tube comprises a more rigid material such as a silicone polymer, the catheter may not have a guide wire.

In some embodiments, at the proximal end of the catheter the tube comprises a bevelled or oblique tip. In some embodiments, the tip has an angle of 30-60°, preferably about 45°, relative to the major axis of the tube. The oblique or bevelled tip can ease the insertion of the catheter into a blood vessel. In some embodiments, at the proximal end of the catheter the tube comprises a rounded tip.

In some embodiments, the marker comprises a marker ring. In some embodiments the marker ring is adjacent to the proximal end of the catheter.

In some embodiments, the catheter comprises an inflatable balloon, preferably provided adjacent to the proximal end of the catheter. In some embodiments, the balloon surrounds the tube. In some embodiments, the catheter comprises a balloon inflation conduit for supplying fluid to the balloon. In some embodiments, the balloon inflation conduit runs along at least part of the interior of the tube. In some embodiments, the balloon inflation conduit has a smaller diameter than that tube. In some embodiments, the balloon inflation conduit comprises a non-return valve. In some embodiments, the balloon inflation conduit is formed in the wall of the tube. Preferably the fluid is an imaging agent as described below. In some embodiments the balloon has a capacity of at least 30 cc, preferably at least 40 cc, more preferably at least or about 50 cc. In some embodiments, the balloon has a diameter when inflated of at least 3 cm, preferably at least 3.5 cm. The balloon is used to at least partially block a blood vessel. This in normally carried out in order to reduce the possibility of the imaging agent flowing into parts of the body other than the heart, for example the head. In some embodiments, the balloon comprises polyisoprene.

In some embodiments, the external surface of the balloon may be modified in order to increase the friction between the balloon and the part of the human or animal body it contacts during use, normally a blood vessel. There are a variety of ways in which the surface of the balloon can be modified in order to achieve this. In some embodiments, the external surface of the balloon comprises one or more protrusions on its surface. In some embodiments, the external surface of the balloon is roughened. This is particularly useful when the catheter is being used on an older person, because their blood vessel walls tend to be smoother.

In some embodiments, at the distal end of the catheter the balloon inflation conduit splits off from the tube. In some embodiments, a reversible direction valve is provided at a distal end of the balloon inflation conduit. A reversible direction valve is a valve which only allows fluid flow in one direction, but this direction can be reversed. In this way, the balloon can be inflated without backflow of fluid, and the direction of the valve can be reversed when desired to allow deflation of the balloon.

In some embodiments, at the distal end of the catheter the tube is provided with a closable tap, optionally a three-way tap. The tap is preferably shaped to accept various standard medical syringes and fittings. The tap can be opened to allow delivery of the imaging agent through the tube, and then closed to prevent backflow of imaging agent and body fluids such as blood.

In some embodiments, at least one marker comprising a substance such that its position once inserted into the body is detectable from outside the body is provided adjacent to or at either a proximal or distal end of the balloon. In some embodiments, the balloon is provided with two markers, one at its proximal end and one at its distal end. In some embodiments, the marker(s) are in the form of a ring.

In some embodiments, the tube comprises at least one, preferably two, side holes in the wall of the tube. In some embodiments, the tube may comprise more than two side holes in the wall of the tube. In some embodiments, the side holes are in the form of a mesh. In some embodiments, the side holes are positioned between the proximal end of the catheter and the proximal end of the balloon. The side holes can assist in dispersing the imaging agent in the body, particularly into the coronary arteries.

In some embodiments, the catheter comprises a valve which is slidably fitted on the tube. In some embodiments, the valve comprises a diaphragm valve. In some embodiments, the valve comprises an external annular groove which is shaped to accept a suture, for example a drawstring suture. In some embodiments, the exterior of the valve is broadly cylindrical. In some embodiments, the exterior of the valve has a diameter which tapers towards the proximal end of the catheter. In some embodiments, the exterior of the valve is cone-shaped. In some embodiments, the valve has an axial tubular hole which closely fits around the tube. In some embodiments, the diaphragm valve is annular and is provided in the axial tubular hole such that it forms a seal between the valve and the tube. By tying a suture on the blood vessel into which the catheter has been inserted, the possibility of material, such as blood or imaging agent, leaking from the blood vessel is reduced.

In some embodiments, the marker(s) and/or guide wire are detectable by computer tomography (CT), preferably multi-detector computed tomography (MDCT), and/or magnetic resonance imaging (MRI). In some embodiments, the marker(s) and/or guide wire comprise a radio-opaque material.

According to another aspect of the invention there is provided a method for post-mortem in-situ cardiac angiography of a human or animal body, the method comprising the steps of:
(a) inserting a catheter into a blood vessel suitable for providing access to the heart of the human or animal body,
(b) pushing the catheter along the blood vessel to a position in which an imaging agent can be delivered through the catheter to the heart,
(c) delivering the imaging agent through the catheter to the heart, and
(d) imaging the heart.

The phrase “in situ” is understood by those skilled in the art to mean that the method is carried out without removing the heart from the body.

In some embodiments, the method comprises an additional step before step (a) of making an incision in the skin of the human or animal body. This is normally carried out in order to provide access to the blood vessel into which the catheter is to be inserted. In some embodiments, the blood vessel is an artery or a vein. In some embodiments, the blood vessel is the carotid artery or the jugular vein.

In some embodiments, the method comprises an additional step, between the steps of making the incision and inserting the catheter, of dissecting the blood vessel from surrounding soft tissue. This is normally carried out in order to provide better access to the blood vessel.

In some embodiments, the method comprises an additional step, before step (a), of forming an at least partial seal in the blood vessel into which the catheter is to be inserted. In some embodiments, the seal is formed by tying off the blood vessel, for example by tying a piece of string or thread around the blood vessel. This is normally carried out in order to reduce the possibility, for example in step (c), of the imaging agent flowing into parts of the body other than the heart, for example the head.

In some embodiments, the method comprises an additional step, before step (a), of making an incision in the blood vessel into which the catheter is to be inserted. The catheter is then inserted into the blood vessel through the incision. If a seal has been formed in the blood vessel, the incision in the blood vessel is made at a point in the blood vessel between the seal and the heart.

In some embodiments, the catheter has a proximal end which is inserted into the blood vessel, and a distal end which remains outside the blood vessel. In some embodiments, step (b) includes pushing the proximal end of the catheter along the blood vessel until the proximal end is in the ascending aorta. In some embodiments, step (b) includes pushing the proximal end of the catheter along the blood vessel until the proximal end is above the aortic valve and adjacent to the coronary ostia.

In some embodiments, the method comprises an additional step, before step (b), of inserting a guide wire along the catheter. The guide wire is preferably comprises a substance whose position in the body is detectable from outside the body, such as titanium. This step is normally carried out in order to provide the user of the catheter with information on the position of the catheter within the body.

In some embodiments, the catheter has a balloon provided on an external surface. In some embodiments, the method comprises an additional step, before step (c), of inflating the balloon. In some embodiments, the balloon substantially blocks the blood vessel into which the catheter is inserted. In some embodiments, the balloon substantially blocks the ascending aorta. Balloon inflation is normally carried out in order to reduce the possibility of the imaging agent flowing into parts of the body other than the heart, for example the head.

The imaging agent can be any substance whose position in the body is detectable from outside the body. In some embodiments, the imaging agent is selected from air, a lipid-based contrast agent or a water-based contrast agent. Suitable lipid-based imaging agents include polyethylene glycol and iodized oil. In some embodiments, the water-based imaging agent comprises a mixture of sodium amidotrizoate (sodium diatrizoate) and meglumine amidotrizoate (meglumine diatrizoate), preferably in a ratio of sodium amidotrizoate:meglumine amidotrizoate of 1:6.6. In some embodiments, the water-based imaging agent is Urografin® (Bayer Healthcare).

In some embodiments, the method of imaging is selected from computer tomography (CT), preferably multi-detector computed tomography (MDCT), and/or magnetic resonance imaging (MRI).

This invention will be further described by reference to the following Figures which are not intended to limit the scope of the invention claimed, in which:

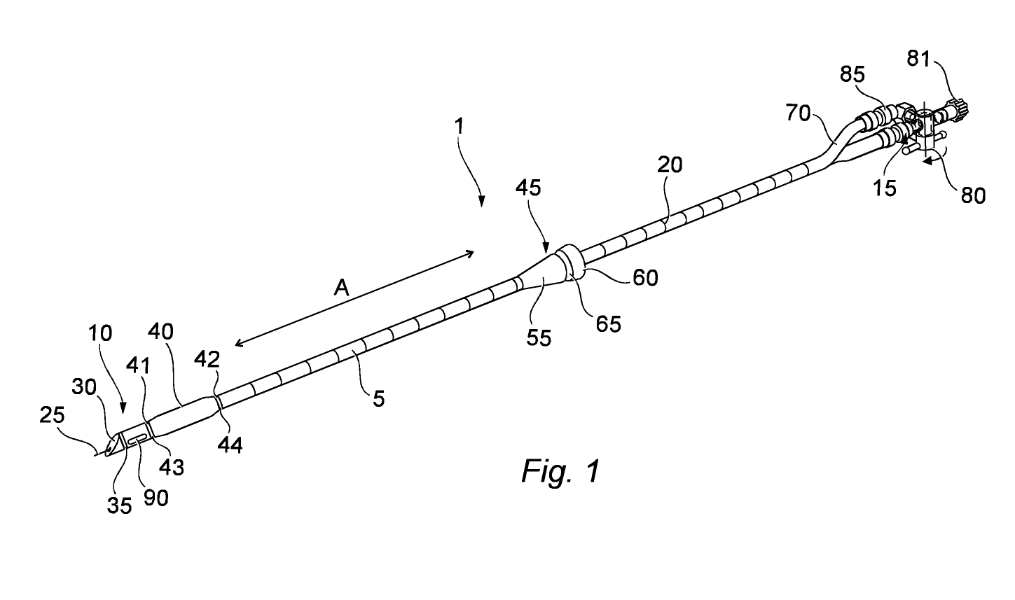

FIG. 1 shows a catheter according to one embodiment of the invention.

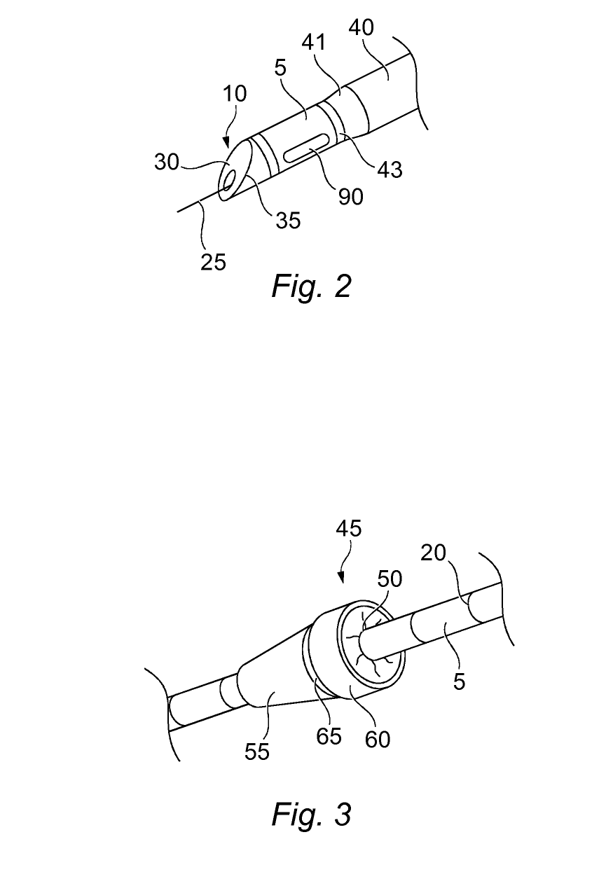

FIG. 2 shows an enlarged view of the proximal end of the catheter shown in FIG. 1.

FIG. 3 shows an enlarged view of the slidable valve of the catheter shown in FIG. 1.

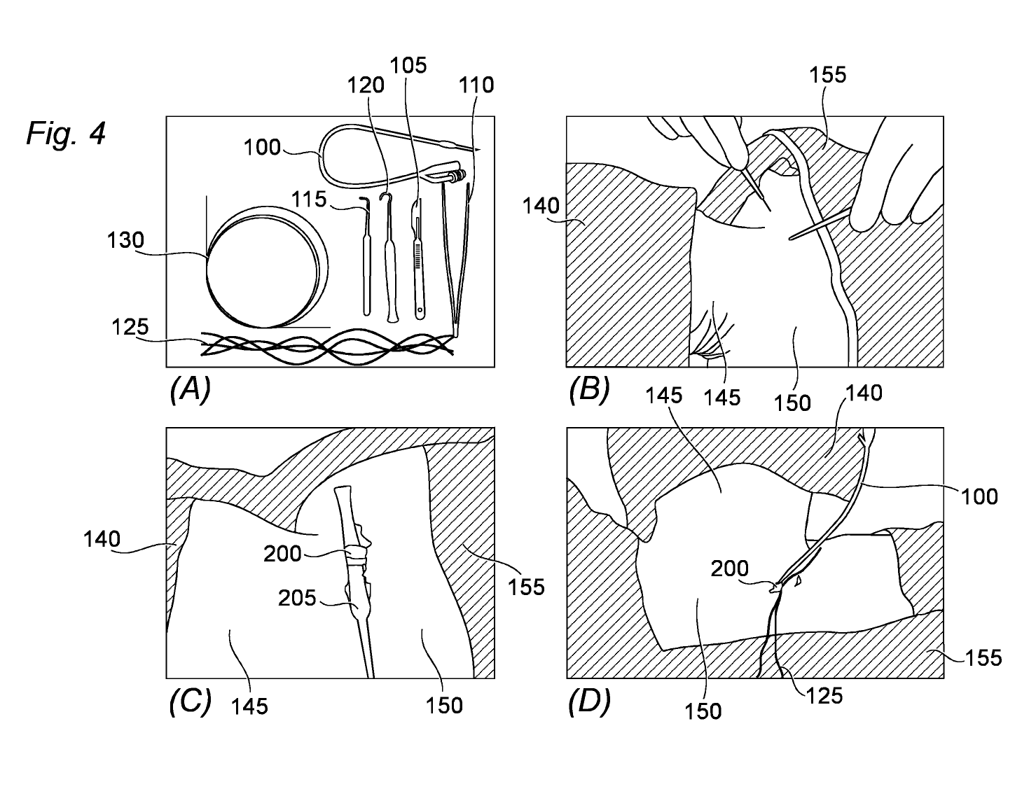

FIG. 4A shows basic mortuary equipment used for cannulation prior to angiography in accordance with one embodiment of the method of the invention.

FIG. 4B shows a supraclavicular incision in accordance with one embodiment of the method of the invention.

FIG. 4C shows the left carotid artery is elevated by means of an aneurysm hook in accordance with one embodiment of the method of the invention.

FIG. 4D shows a Foley catheter inserted into the carotid artery in accordance with one embodiment of the method of the invention.

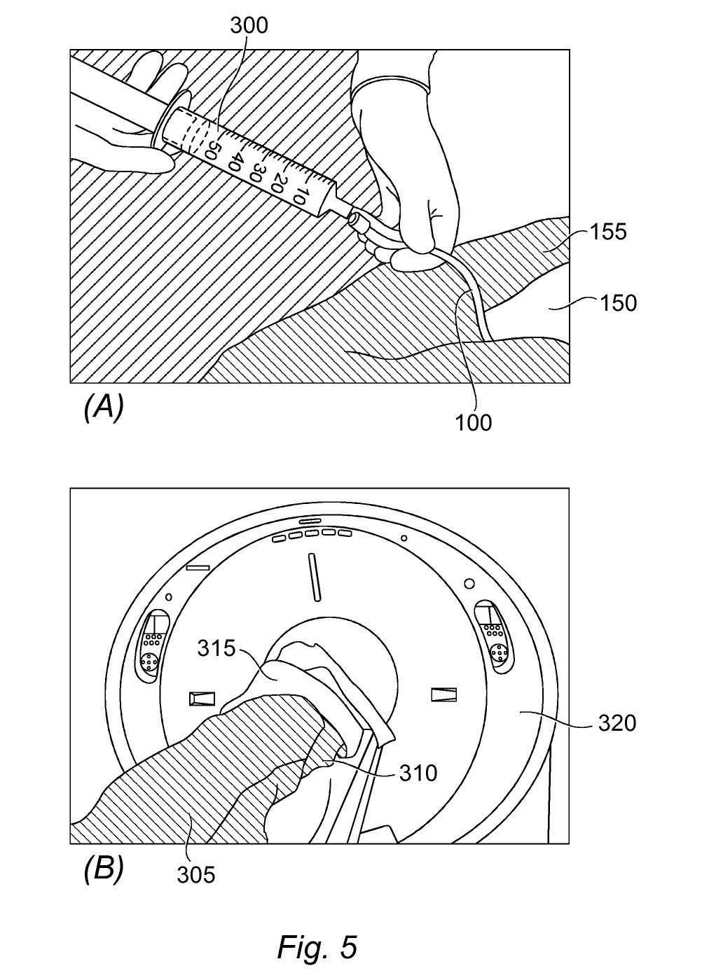

FIG. 5A shows the injection of contrast agent into the catheter in accordance with one embodiment of the method of the invention.

FIG. 5B shows a body in the right lateral decubitus position in accordance with one embodiment of the method of the invention.

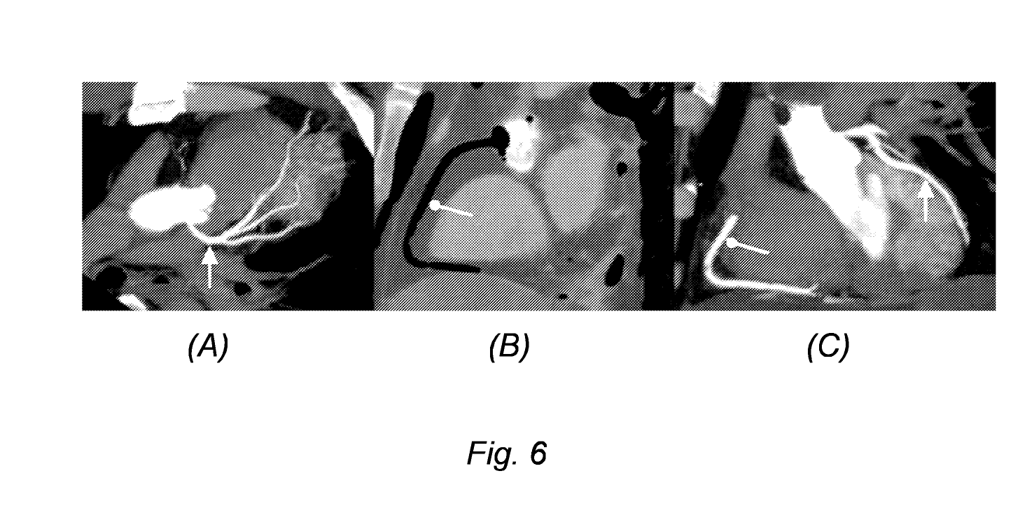

FIGS. 6A, 6B and 6C show images from various post-mortem coronary angiographies with normal vessel anatomy and luminal patency.

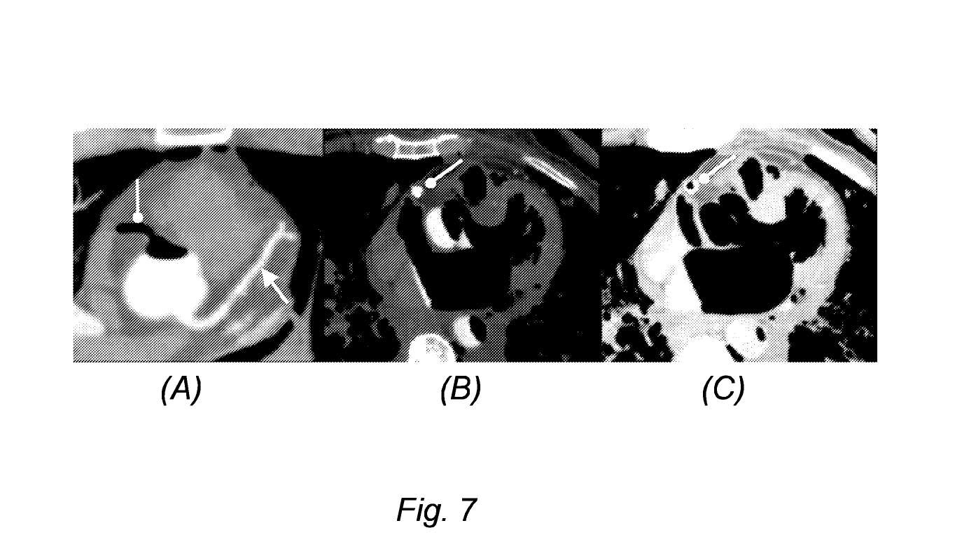

FIGS. 7A, 7B and 7C show images from different post-mortem coronary angiographies using positive and negative contrast agents.

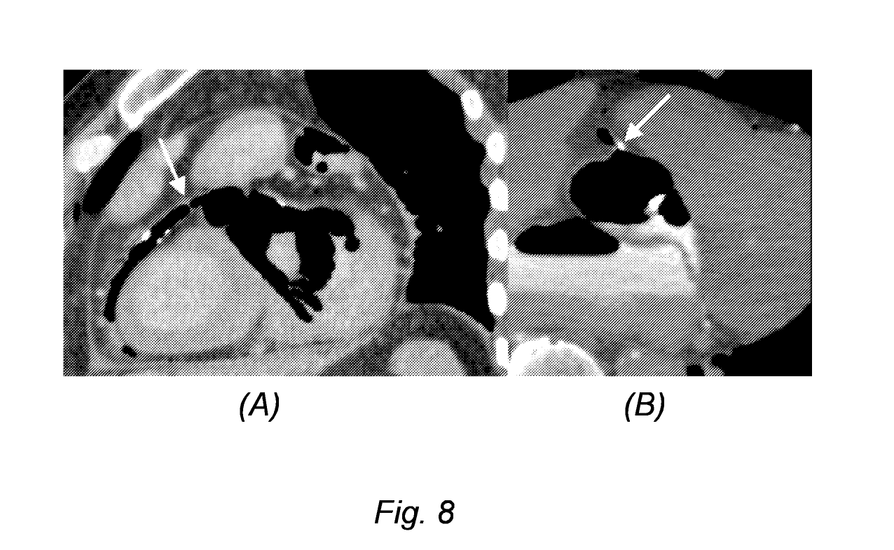

FIGS. 8A and 8B show images from different post-mortem coronary angiographies with mild calcified plaque disease and moderate ostial stenosis.

FIGS. 1-3 depicts a catheter according to one embodiment of the invention. As shown in FIGS. 1 and 2, the catheter 1 comprises an open-ended tube 5. The catheter has proximal end 10 for insertion into a human or animal body on which post-mortem angiography is to be carried out. As shown in FIG. 1, the catheter 1 also has a distal end 15 which remains outside the human or animal body during normal use.

The catheter 1 would normally be a non-sterile multi-use device whose shelf life and multi-use is dependent upon the material used to manufacture the device. The catheter 1 can also be single use if required.

The tube 5 is made from a flexible polymer, for example a silicone polymer or latex. Since the catheter 1 is for post-mortem use, there is no concern about the possibility of causing an allergic reaction in the body into which it is to be inserted. However, an allergic reaction is a consideration for the user of the catheter 1. A suitable tube 5 is a 14Fr Foley catheter tube.

As shown in FIG. 1, the tube 5 has graduations 20 marked along its length. The graduations are spaced 1 cm apart and are provided in order to give the person carrying out the post-mortem angiography an indication of how far the catheter 1 has been inserted into a human or animal body on which post-mortem angiography is to be carried out. The graduations 20 can also assist the user in repositioning the catheter 1 during angiography.

In the embodiment shown in FIGS. 1-3, a guide wire 25 extends along the length of the interior of tube 5 and is shown in FIGS. 1 and 2 protruding from proximal end 10 of catheter 1. In other embodiments of the invention, if the tube 5 comprises a sufficiently rigid material the guide wire may be omitted. The guide wire 25 can be formed of any material known in the art such that the guide wire 25 can be viewed using an imaging system such as MDCT or MRI once the catheter 1 has been inserted into a human or animal body on which post-mortem angiography is to be carried out. A suitable material for the guide wire 25 is titanium. The guide wire 25 preferably flexible. The guide wire 25 is normally 20-30 cm longer than the catheter 1.

As shown in FIGS. 1 and 2, the proximal end 10 of catheter 1 the tube 5 has a bevelled or oblique tip 30 which is angled at approximately 45° to the axis A of tube 5. The bevelled tip 30 is provided in order to ease insertion of the proximal end 10 of catheter 1 into a human or animal body on which post-mortem angiography is to be carried out.

As shown in FIGS. 1 and 2, the proximal end 10 of catheter 1 is provided with marker ring 35. Marker ring 35 can be formed of any material known in the art that is viewable using an imaging system such as MDCT or MRI once the catheter 1 has been inserted into a human or animal body on which post-mortem angiography is to be carried out. The marker 35 provides to the person carrying out the post-mortem angiography an indication of the position of bevelled tip 30 inside the body.

FIG. 1 shows an inflatable balloon 40 provide close to proximal end 10 of catheter 1, normally about 1 cm from the tip 30. The balloon 40 surrounds tube 5 and is normally made from a silicone polymer or latex. Although not shown in FIGS. 1 and 2, the external surface of the balloon 40 is provided with protrusions on its external surface.

The balloon 40 has proximal and distal ends 41,42. Balloon markers 43,44 are provided on tube 5 at proximal and distal ends 41,42 of balloon 40. Balloon markers 43,44 can be formed of any material known in the art such that it can be viewed using an imaging system such as MDCT or MRI once the catheter 1 has been inserted into a human or animal body on which post-mortem angiography is to be carried out. The balloon markers 43,44 provide to the person carrying out the post-mortem angiography an indication of the position of balloon 40 inside body.

Although not shown in the Figures, the catheter 1 has a balloon inflation conduit for inflating and deflating balloon 40. The balloon inflation conduit extends distally from balloon 40 along the interior of tube 5. The balloon inflation conduit can be formed by dividing off a section of the interior of tube 5, or it can be formed by tube 5 surrounding or being surrounded by a second tube.

As shown in FIG. 1, at the distal end 15 of the catheter 1 the tube 5 has an arm 70. Arm 70 branches off and then runs parallel to tube 5 and contains the balloon inflation conduit. Arm 70 is provided at proximal end 15 with reversible direction valve 85 for supplying fluid (liquid or gas) to inflate the balloon 40 or for withdrawing fluid to deflate the balloon 40, as well as for maintaining balloon inflation. A three-way tap may be used instead of reversible direction valve 85.

At the distal end 15 of the catheter 1, the tube 5 has a three-way tap 80 attached to it, the proximal end of which is provided with cap 81 which seals the tube 5. Fluid imaging agents are injected through tap 80. Tap 80 controls injection and can prevent back flow. Tap 80 is shaped to accept standard medical syringes and fittings.

As shown in FIGS. 1 and 2, in addition the opening at tip 30, the tube 5 is provided with side holes 90 (only one of which is visible in the Figures) in the wall of the tube 5. The side holes 90 can assist in dispersing the imaging agent in the body. The side holes 90 are normally positioned about 0.5-1 cm from the tip 30.

As shown in FIGS. 1 and 3, a diaphragm valve 45 is slidably fitted onto the tube 5 between the balloon 40 and the distal end 15 of catheter 1. The valve 45 comprises an axial tubular hole 50 (see FIG. 3) which closely fits around tube 5. The valve 45 also comprises proximal annular bevel 55 and a distal annular section 60 which are spaced apart by annular groove 65. The annular groove 65 is shaped to accept a drawstring suture, which is used to seal a blood vessel (not shown) into which the catheter 1 is inserted so that material does not leak out of the blood vessel during use.

Adults and paediatric versions of the catheter 1 are envisaged, as are catheters 1 suitable for use on small and large animals. A standard adult catheter 1 would be about 30 cm long, with a 14Fr tube and a 50 cc balloon.

In use, an incision is made in skin of the human or animal body on which the post-mortem cardiac angiography is being carried out in order to access a blood vessel suitable for accepting the catheter 1. In humans, the incision is normally made in the neck. The blood vessel is normally the carotid artery.

The carotid artery is then dissected free of soft tissue. A seal is then made, for example by tying a piece of string around the artery, to prevent the imaging agent to be administered through the catheter 1 from flowing into the head of the human or animal body. An incision is then made in the part of the carotid artery between the seal and the heart, and which is dissected free of soft tissue.

The proximal end 10 of catheter 1 is then inserted into the incision and pushed along the carotid artery until the bevelled tip 30 is in the ascending aorta, above the aortic valve and adjacent to the coronary ostia.

Valve 45 is then slid along tube 5 such that it blocks the incision made in the carotid artery. The valve 45 is then held in place by a drawstring suture, the drawstring suture being tied such that it is accepted by annular groove 65 of valve 45.

The balloon 40 is then inflated by supplying fluid, in this embodiment air, through reversible direction valve 85, along the balloon inflation conduit and into balloon 40. The balloon 40 preferably has a capacity of 50 cc so that inflation results in the ascending aorta being substantially blocked by the inflated balloon 40.

An imaging agent is then supplied through tap 80 and along tube 5 such that in flows out from proximal end 10 and into the blood vessels of the heart (not shown) of the human or animal body on which the post-mortem angiography is being carried out.

FIG. 4A shows basic mortuary equipment used for cannulation prior to angiography. This equipment includes a 14 Fr silicone male Foley Catheter 100; a scalpel 105; toothed forceps 110; two aneurysm hooks 115,120; two lengths of string 125 and a standard radiographic guide wire 130.

FIGS. 4B, 4C and 4D shows a supraclavicular incision made on the left side of a human chest 150, based on a standard embalming technique. The person's face is covered by cloth 140, their neck 145 and chest 150 are exposed, and the rest of their body is covered by sheet 155.

Following blunt dissection of the soft tissue, the left carotid artery 200 is elevated by means of an aneurysm hook 205 prior to incision of the anterior arterial wall (see FIG. 4C). FIG. 4D shows the Foley catheter 100 being inserted into the carotid artery 200. Pieces of string 125 are used to tie off the superior aspect of the artery and as a marker for the inferior aspect.

FIG. 5A shows the injection of a contrast agent (in this case air) into the Foley catheter 100 by means of a 60 ml syringe 300 whilst on the CT scanner table (not shown). Again, the person's chest 150 is exposed, and the rest of their body is covered by sheet 155. FIG. 5B shows the sheet-covered human body 305 rolled 90° to the right lateral decubitus position (whilst still in the body bag) and held in place with foam blocks 310 and securing straps 315, ready to undergo an scan using MRI machine 320.

FIG. 6A-C shows images from various post mortem coronary angiographies showing normal vessel anatomy and luminal patency. FIG. 6A shows a curved MPR, minimum intensity projection image showing normal left coronary system (arrow). FIG. 6B shows a curved MPR, minimum intensity projection image showing normal RCA (arrow with round head). FIG. 6C shows a curved MPR, minimum intensity projection image showing normal left (arrow) and right coronary artery segments (arrow with round head).

FIG. 7A shows preferential filling of RCA (round head) with air and LAD (arrow) with contrast. Luminal delineation of coronary artery in the presence of calcified plaque is inferior with positive (FIG. 7B) compared to negative contrast (FIG. 7C).

FIG. 8A shows a curved MPR image (a) showing proximal RCA stenosis (arrow) with mild calcified plaque disease distally. Axial image (FIG. 8B) shows moderate ostial stenosis of the RCA due to a mixed plaque disease (arrow).

Detailed Discussion of Trials

Trials were conducted within local guidelines with approval by the local research ethics committee (LREC 04/Q2501/64, UHL 09523) and supported by the local Coroners' offices. Cases for the first part of the study involving the development of the cadaver angiography method were selected for potential coronary angiography by the inventors from the routine coronial post-mortem request forms faxed to the Leicester Royal Infirmary mortuary. Exclusion criteria included significant neck trauma (to ensure method was assessed on normal individuals), previous complex cardiac surgery such as bypass grafting (for the same reason) and bodies weighing over 125 kg (due to body handling issues). Cases with a short post-mortem interval were selected where possible. The next of kin were contacted by the Trial Consenter (an Advanced Nurse Practitioner with counselling experience) on the day prior to the autopsy. Informed consent was obtained by telephone with information leaflets being sent to next of kin the next working day.

To develop the method for cadaver MDCT angiography is was necessary to consider and try to overcome a number of issues as discussed below:

Site And Method of Vascular Access

All canulations were performed within a Human Tissue Authority licensed mortuary. The first consideration was site selection for access to the vascular system. A number of sites were considered, including the femoral, axillary and subclavian arteries. The carotid artery was chosen due to its lack of branches making it easier to raise. It is also the vessel closest to the arch of the aorta allowing a shorter catheter to be selected. The method for raising the carotid artery was adapted from standard embalming techniques, and used standard mortuary equipment (FIG. 1a). Access to the carotid artery initially involved an oblique incision to the neck, approximately 5 cm above the clavicle to the lateral side of the neck. The subcutaneous tissue was blunt dissected with an aneurysm hook. The soft tissue was divided down inferior to the left sternocleidomastoid muscle. The jugular vein and vagus nerve were dissected free and pulled laterally (a string sling can be used to facilitate this). The soft tissue was further dissected inferiorly and medially until the carotid artery was located. The carotid artery was dissected free of the soft tissue and elevated by means of an aneurysm hook. Once raised, a piece of string was passed under the artery and used to tie off the most superior aspect of the artery to prevent flow of contrast into the head. A second piece of string was threaded under the artery and pulled inferiorly and loosely tied to act as a sling or a marker for the lower aspect of the artery. The artery was held in place during this process by threading the aneurysm hook underneath the artery. A horizontal cut was made across the anterior wall of the artery. The posterior wall was left intact, to prevent retraction of both ends of the artery. The lower cut aspect of the carotid artery wall was held with toothed forceps, and the catheter inserted. The aim was to place the bevelled tip in the ascending aorta, just above the aortic valve and adjacent to the coronary ostia. A balloon could then be inflated in the ascending aorta above this. During the refinement of the method, both carotid arteries were accessed to assess the better side to develop the final protocol.

Catheter Choice

Different types of catheter available within medical practice were tried over the first 12 cases, including a variety of pigtail angiographic catheters, large balloon size catheters including an endovascular aneurysm repair (EVAR) balloon catheter and a Trans-Catheter Aortic Valve Implantation (TAVI) balloon catheter. These were initially chosen for their directional capability, as it was thought that this would be useful to navigate the aortic arch and ensure correct placement into the ascending aorta.

After trialling these catheters, a 14 Fr silicone coated male urinary catheter (Bardia Foley Catheter) with standard guide wire (Cook, fixed core wire guide: straight) was selected. A guide wire was initially used to determine the catheter position on MDCT, rather than to facilitate introduction. The balloon was inflated with water initially, but this was changed to dilute water-soluble radiographic contrast (1 in 50 dilution of Urografin®) to ascertain the position of the balloon in the aorta on localiser scans. This removed the necessity for the use of the guide wire 25 except in difficult cases.

Contrast Medium Choice

The literature relating to post-mortem angiography describes a number of different contrast agents being used, each with its own advantages and disadvantages. The Virtopsy® group advocate the use of lipid based contrast agents (polyethylene glycol and iodized oil) in conjunction with the use of a modified heart-lung bypass machine to create a ‘circulation’. This process is complex and appears time consuming. A quick and simple method that could be used in any mortuary, be it permanent or temporary, in any country, using readily available materials requiring minimal training was sought. Furthermore, it was desired to use contrast media known to have little physiological impact in the extravascular extracellular space as this could affect any subsequent histological examination. Water-based contrast agents have been reported to cause tissue oedema leading to significant artefact generation on CT. This appears to increase if there is a long delay between the injection of the contrast and the completion of the scan. Delays such as this are not applicable to this study. Water-soluble iodinated contrast media Urografin® 150 mgl/ml (Bayer Healthcare, approximately £20 per 500 ml bottle) was used. A 1 in 10 dilution was used, which was calculated to be similar to concentrations achieved in clinical CT coronary angiography.

As vessels are often well delineated by the development of air in the post-mortem period, injected air was used as a ‘negative’ contrast agent. This was hypothesised to be potentially superior to ‘positive’ contrast agents due to better delineation of areas of calcification. Using repeated injections with a 60 ml syringe a variety of volumes and infusion rates of air and Urografin® were researched.

Scanning Protocol

The MDCT scan was undertaken under a standard post-mortem protocol using a Toshiba Aquilion 64 slice scanner (120 kVp, 300 mA and 64×0.5 mm slice thickness, matrix 512×512) reconstructed to either 1 or 2 mm thick slices. Cardiac images were then performed with a narrow field of view covering the heart and aortic arch with 1 mm slice reconstructions pre and post contrast injection. Scan time was recorded for the final 14 cases (after the initial learning curve). Times were recorded as whole scan time (from arrival to leaving the CT scan room) and were divided into two sections relating to the time to complete the standard CT scan and the extra time of the angiography.

Positioning of the Body

All the bodies were initially scanned within a labelled body bag in the supine position with the arms at either side. Bodies were scanned at variable post-mortem intervals and in various states of rigor mortis. Access to the body was provided through an opening at the head end of the body bag for the angiography.

As previous studies have used rotation of the body to assist contrast agent dispersal, different body positions were experimented with during injection. The positioning of the body was facilitated by foam blocks and secure strapping (standard radiographic equipment). The body was handled within the body bag so there was no risk of contamination of the scanner suite with body fluids.

Evaluation of Angiography

Cardiac imaging was reported by two radiologists, one a Consultant Cardiac Radiologist and the other with five years experience of post-mortem imaging. Image analysis was performed on workstations using multiplanar reconstructions (MPR) and 3-D analysis. The optimum filling of the vessels by either air or positive contrast media was assessed subjectively on the ability to see the right coronary artery (RCA), posterior descending artery (PDA), left main stem (LMS), left anterior descending (LAD) and circumflex (Cx) arteries by positive or negative contrast. The initial cases were used to improve the protocol. A more formal assessment of artery delineation was undertaken for the final 10 cases, although minor changes were still being made to the protocol. Consistent with clinical practice and standardised cardiac dissection at autopsy, intramural vasculature was not assessed in this part of the study. After 25 cases a final protocol was agreed in order to evaluate the use of CT coronary angiography in post-mortem investigation.

Results

A total of 25 cases were recruited to develop the final present method and protocol.

Cannulation

After feedback from the Anatomical Pathology Technologists (APTs) that incisions higher up on the side of the neck were difficult to conceal with a standard shroud, the approach was modified to a lower one based on the antero-lateral (supraclavicular) embalming technique (FIG. 4B). An almost horizontal incision was made above the clavicle on the inferior aspect of the neck and the artery was raised as before (FIG. 4C). An additional benefit of this new approach was that the catheter insertion into the ascending aorta was easier than further up the neck as manipulation of the artery was easier, allowing the vessel to be pulled laterally and inferiorly to align the vessel with the arch of the aorta (FIG. 4D). The visual exposure of the vessel at this location was better, and allowed easier repositioning of the catheter in the scan room if required.

Both sides of the neck were used in 7 cases, the right only in 6 and the left side only in 12. In 5 out of 13 cases there was failed access to the ascending aorta using the right approach and in 1 out of 9 cases using the left sided approach. The left sided approach therefore had a significantly higher success rate (chi-squared test p=0.02). The single failure on the left side was due to inadvertent cannulation of the internal jugular vein, a mistake that was considered unlikely to be repeated. For the last 12 cases only the left side was accessed and a guide wire was used on only one occasion to assist placement of the catheter. With right-sided cannulation there were problems with advancing the catheter out of the carotid artery, into the aortic arch and down the ascending aorta. The catheter would often hit the inferior wall of the aortic arch and fail to advance or go down the descending aorta. Access to the ascending aorta from the right side could be achieved by an Interventional Radiologist with guide wires and angled catheters, but this is time consuming compared to the easier access from the left side.

Catheter

Angiographic guide wires and catheters were initially used to aid manipulation of the catheter position. These catheter systems were used with a rigid ‘introducer’; however, it was found that it caused damage (specifically perforation of the carotid artery in one case and of the aortic arch in a second). It was found to be relatively easy to introduce the guide wire or catheter from the left sided approach without the introducer, therefore its used was abandoned early on. The catheter system was time consuming and it was found that, if access was adopted via the left carotid artery just above the level of the clavicle, the directional capability was unnecessary.

It soon became evident that the most important part of the catheterisation was the balloon inflation in the ascending aorta to prevent flow back down the descending aorta. Balloon inflation below 3 cm in diameter proved unsatisfactory. Due to the easier access experienced from the left sided approach it was possible to change to a silicone coated 14 Fr Foley male urinary catheter to allow larger balloon inflation. To allow the use of a guide wire an oblique incision to the rounded tip of the catheter was made. This modified tip assisted catheter introduction into the artery. The flexibility of the catheter, along with the length and inflatable capacity of the balloon, was found to be adequate. Latterly the guide wire was used only for difficult cases.

In the early cases it was found to be difficult to ‘feel’ whether the catheter was positioned correctly and a CT scan was relied upon to determine this. As the operators became more experienced it was possible to confidently feel whether the catheter was in the correct position. If the catheter contacted the inferior wall of the arch of aorta, a very clear obstruction to the advancement of the catheter was felt. If it was possible to insert the entire length of the catheter without meeting any resistance, it was more likely that the catheter had gone down the descending aorta and repositioning was required. If the catheter went down the ascending aorta, the catheter would inevitably contact the leaflets of the aortic valve, producing a sensation of a ‘bounce’ after advancing 10-15 cm. The process of learning this haptic feedback (‘to feel the bounce’) is a key stage in order to perform the technique independently. Catheter position was confirmed using the initial chest CT scan, and was modified as above and confirmed with a repeat scan if necessary.

No problems were experienced with catheter movement during the transportation of the body from the mortuary to the CT scanner. A simple piece of adhesive tape across the end of the catheter secured to the side of the neck was sufficient to prevent movement. A towel could be folded and wrapped around the neck to absorb any leakage of blood during transportation. A second towel could be wrapped around the patient's head, hiding the face to preserve patient dignity and prevent any distress to the radiographers.

Contrast Injection

Scanning was carried out immediately after injecting the contrast and did not see any artefacts related to the contrast. The injection of Urografin® did not cause any damage to the vessels or organs detectable at autopsy. As a targeted system was used, the contrast did not alter post-mortem toxicology as it entered the heart and aorta only. It did not cause the artefactual fat emboli seen when using other contrast types and would not interfere with the diagnosis of air embolus as the right side of the heart and pulmonary arterial vessels are not investigated by this approach. This means that there is no need to take samples from the body prior to angiography being performed.

In the early cases using the angiographic catheters 60 ml manual injections of positive contrast at varying speeds from 5 to 20 seconds (FIG. 5A-C) were tried. It was found that the slower manual injections with continued pressure were much better at circulating the contrast than the fast, high-pressure injections. After a few test cases larger volumes were administered giving better images. No significant improvement was found in positive contrast filling of the vessels over 120 ml. The final protocol was for manual injection of 120 ml of positive contrast in 2 injections in approximately 40 seconds (20 seconds per syringe).

Urografin® contrast agent is of the same opacity as the calcification seen within the wall of the coronary arteries making assessment of vessel lumen difficult. Also, positive contrast filling of the RCA and PDA was often incomplete but the vessels filled well when air was used (see below). The former could be well demonstrated by 3-D multiplanar reconstruction (MPR) by maximal intensity projection (MIP) while the latter required minimum intensity projection (FIG. 6A-C). Air had the distinct advantage of showing a patent lumen in the middle of a calcified plaque (FIG. 7A-C). With this in mind a double contrast method was adopted; an initial injection with air followed by a further injection of Urografin®. For the first few cases air was injected in 60 ml increments until good filling was achieved. The final protocol was for 300 ml of air in 5 manual injections using a standard 60 ml bladder syringe using gentle constant hand pressure over a period of 2 to 3 minutes.

Body Position

Initially, the body remained in the supine position throughout all stages of contrast injection. Injection of air in the supine position resulted in consistent filling of the RCA, but not the LMS and its branches. Likewise positive contrast media filled the left sided branches, but not the RCA (FIG. 7A-C). The body was inclined 90° to the right lateral decubitus position as this was shown to improved filling of the RCA with positive contrast and filling of the LMS, LAD and Cx with air (FIG. 5). Contrast injection was not performed in the right lateral decubitus position as it was found that filling of the RCA with air and contrast was sufficient with the above body position.

Pathology

One concern is that injection of air/Urografin® may dislodge thrombi, misplacing them for autopsy study or open stenosed segments (FIG. 8A-B). One case showed a complete occlusion of the LAD throughout the air and contrast studies, and at subsequent autopsy it was shown to be an occlusive thrombus. Although a single case, it perhaps negates the above concern. Correlation of CT angiography with post mortem findings is being carried out.

Timing

The average time for cannulation was 15 mins (range 5-30 minutes). The longer time periods reflect the earlier cases and the initial learning period for the technique. In later cases, when the operators were confident with the technique, the cannulation could be performed in approximately 5 minutes with minimal blood spillage using standard mortuary equipment. Certain cases proved more problematic for cannulation such as obese patients with short fat necks and cases with advanced rigor where the neck was flexed in an abnormal position.

For the final 14 cases whole scan time ranged from 39 to 90 minutes (mean 60 minutes). The standard post-mortem imaging time was 18 to 40 minutes, mean 28 minutes and the angiography time was 15 to 55 minutes (mean 32 minutes). For the last 6 cases the whole scan time improved to 39 to 63 minutes (mean 48 minutes).

Discussion

The above discussion outlines the developmental stages and final method for targeted in-situ post-mortem cardiac angiography. The cannulation can be performed with easily accessible autopsy/hospital equipment in a permanent or temporary mortuary by an APT or pathologist with minimal training. The CT scan can be performed by suitably trained radiographer or APT. Ideally the operator should be able to assess the success of the angiography at the time of the scanning and whether further air or contrast injections are required. The operator can be any suitably trained individual. In this trial it was either a radiologist or a pathologist but it could be a radiographer or APT. At the beginning of the trial the pathologists involved had a poor understanding of cardiac CT axial anatomy and disease. However, following a period of teaching under the supervision of a radiologist the pathologists were confidently able to assess catheter position and vessel filling hence it is asserted that any suitably trained health care professional could undertake this work. Thus, after the first 10 cases a radiologist was no longer required to be present at the time of scanning.

Although the coronary vessels could often be opacified completely by air, positive contrast has been used as well. This is because the presence of positive contrast in the distal vessels is a good sign of patency in clinical practice whereas distal air may simply relate to post-mortem changes.

With increasing experience it was found that the time for post-mortem whole body scanning including coronary angiography reduced to an average of 48 minutes. It is anticipated that mortuary preparation time will eventually be less than 10 minutes and the entire period in the scanning suite less than 45 minutes.

Evaluation of CT scans in comparison to autopsy findings form part of the ongoing study and are not presented here. It was however, essential that a standardised protocol be developed at the beginning of this trial to enable visualisation of the coronary arteries prior to the next stage of the project.

Described herein is a simple, quick, cost effective, manual, and targeted in situ post-mortem cardiac angiography catheter and method using a minimally invasive approach, to be used with MDCT for high throughput cadaveric imaging in permanent or temporary mortuaries. It is believed that the protocol described below can be adopted by practitioners working in any mortuary to obtain diagnostic images of the coronary arteries. Ongoing research is still needed to evaluate whether cardiac MDCT is comparable to current autopsy practice. However, the use of methods such as presented in this paper will assist those considering this problem.

An example protocol for angiography is set out below:
1. Consent
2. Cannulation of left common carotid artery at the suprclavicular location in the mortuary
3. Transport body to CT scanner
4. Scout and system scanning and catheter positioning scan
5. Angiography:
   a. Ensure correct placement of catheter
   b. Body supine: Inject 300 ml air, scan and evaluate.
   c. Body rolled into right lateral decubitus position: Inject 300 ml air, scan and evaluate
   d. Body rolled back to supine: Inject 120 ml Urografin®, scan and evaluate.
6. Transport body back to mortuary in preparation for autopsy (if required)
7. Images reported independently by Radiologist(s).

1. A catheter for use in post-mortem angiography, the catheter comprising:
(a) a proximal end for insertion into a human or animal body,
(b) a distal end which remains outside the human or animal body during normal use,
(c) a tube for delivering an imaging agent to the human or animal body, and
(d) a marker for insertion into the human or animal body, the marker being positioned at a predetermined distance from the proximal end and comprising a substance such that its position once inserted into the human or animal body is detectable from outside the human or animal body.

2. The catheter as claimed in claim 1, wherein the tube has a diameter greater than or equal to approximately 3 mm.

3. The catheter as claimed in claim 2, wherein the tube has a diameter greater than or equal to approximately 4 mm.

4. The catheter as claimed in claim 1, wherein at the proximal end of the catheter the tube comprises a bevelled or oblique tip.

5. The catheter as claimed in claim 1, wherein the catheter comprises an inflatable balloon.

6. The catheter as claimed in claim 5, wherein a surface of the inflatable balloon is modified in order to increase friction between the inflatable balloon and the human or animal body.

7. The catheter as claimed in claim 5, wherein the balloon has a capacity of at least approximately 30 cc.

8. The catheter as claimed in claim 7, wherein the balloon has a capacity of at least approximately 40 cc.

9. The catheter as claimed in claim 5, wherein the inflatable balloon has a diameter when inflated of at least approximately 3 cm.

10. The catheter as claimed in claim 1, wherein the tube comprises at least one side hole in a wall of the tube.

11. A method for post-mortem in-situ cardiac angiography of a human or animal body, the method comprising the steps of:
(a) inserting a catheter into a blood vessel suitable for providing access to a heart of the human or animal body,
(b) pushing the catheter along the blood vessel to a position in which an imaging agent can be delivered through the catheter to the heart,
(c) delivering the imaging agent through the catheter to the heart, and
(d) imaging the heart.

12. The method as claimed in claim 11, wherein the blood vessel is a carotid artery or a jugular vein.

13. The method as claimed in claim 11, wherein the method comprises making an incision in the blood vessel into which incision the catheter is inserted.

14. The method as claimed in claim 11, wherein the method comprises forming an at least a partial seal in the blood vessel into which blood vessel the catheter is inserted.

15. The method as claimed in claim 14, wherein the incision in the blood vessel is made at a point in the blood vessel between a seal and the heart.

16. The method as claimed in claim 11, wherein the pushing includes pushing a proximal end of the catheter along the blood vessel until the proximal end is in an ascending aorta.

17. The method as claimed in claim 11, wherein the catheter comprises an inflatable balloon.

18. The method as claimed in claim 17, wherein the method comprises inflating the inflatable balloon.

19. The method as claimed in claim 18, wherein the inflatable balloon substantially blocks the blood vessel into which blood vessel the catheter is inserted.

20. The method as claimed in claim 11, wherein the imaging is selected from computer tomography and magnetic resonance imaging.

21. The method as claimed in claim 11, wherein the catheter comprises:
(a) a proximal end for insertion into the human or animal body,
(b) a distal end which remains outside the human or animal body during normal use,
(c) a tube for delivering the imaging agent to the human or animal body, and
(d) a marker for insertion into the human or animal body, the marker being positioned at a predetermined distance from the proximal end and comprising a substance such that its position once inserted into the human or animal body is detectable from outside the human or animal body.
